(19) United States
(12) Patent Application Publication
(10) Pub. No.: US 2018/0360314 A1
(43) Pub. Date: Dec. 20, 2018

(54) LOW-POWER WIRELESS SOLUTION FOR MBAN APPLICATIONS WITH MULTIPLE AGGREGATOR DEVICES

(71) Applicant: KONINKLIJKE PHILIPS N.V., EINDHOVEN (NL)

(72) Inventors: Dong WANG, SCARSDALE, NY (US); Javier ESPINA PEREZ, EINDHOVEN (NL)

(21) Appl. No.: 16/060,579

(22) PCT Filed: Dec. 21, 2016
(86) PCT No.: PCT/EP2016/082247

§ 371 Date: Jun. 8, 2018

Related U.S. Application Data
(60) Provisional application No. 62/270,902, filed on Dec. 22, 2015.

(51) Int. Cl. A61B 5/00; G06F 19/00; H04W 52/02; A61B 5/0205

(52) U.S. Cl. CPC A61B 5/0024; G06F 19/00; H04W 52/0225; A61B 2560/0209; A61B 5/0205; A61B 5/7275; A61B 5/742; A61B 5/0006

(57) ABSTRACT

A Medical Body Area Network (MBAN) (10) includes a hub device (18) with a wireless communication transceiver (34). One or more sensor devices (14) each include a physiological sensor (24) for acquiring physiological data and a wireless communication transceiver (22) configured to connect with the wireless communication transceiver (34) of the hub device (18) to wirelessly transmit acquired physiological data to the hub device (18). One or more aggregator devices (16) each include a wireless communication transceiver (30) for receiving physiological data acquired by the one or more sensor devices (14). The hub device (18) is configured to respond to not wirelessly receiving a missing data portion of physiological data acquired by a sensor device (14) by requesting and receiving the missing data portion from an aggregator device (16).

### FIELD

The following relates generally to wireless communication. It finds particular application in conjunction with medical body area networks (MBANs), and will be described with particular reference thereto. However, it is to be understood that it also finds application in other usage scenarios and is not necessarily limited to the aforementioned application.

### BACKGROUND

There is a general trend in the healthcare industry towards ubiquitous patient monitoring, which is to provide continuous and patient centric monitoring services during the whole care cycle. Such ubiquitous monitoring services can significantly improve quality of care. For example, patient deterioration could be detected at an early stage and early intervention can effectively prevent severe adverse events to happen.

The advance of wireless communication technologies makes such ubiquitous monitoring feasible. The medical body area network (MBAN) technique has been considered as one of the promising solutions to achieve ubiquitous monitoring. A medical body area network (MBAN) replaces the tangle of cables tethering hospital patients to their bedside monitoring units with wireless connections. MBAN is a low-power wireless network of sensors around/on a patient used for monitoring patient's physiological data.

FIG. 1 shows a typical MBAN system. In such MBAN system, several (typically miniature) sensor devices are placed on patient body to capture patient's physiological data, such as heart rate and electrocardiograph (ECG) waveforms, and the captured data is forwarded to a hub device through a short-range and low-power MBAN. The hub device may be a local bedside monitoring unit, cell phone, set-top-box, or other device with wireless connection to the MBAN sensors, and usually has a wired or wireless network connection to a backhaul network (e.g. a third/fourth generation (3G/4G) cellular network, LAN, PAN, WiFi, or the like), through which the collected data can be further transferred to a remote patient monitoring (PM) server. The remote patient monitoring server is responsible for analyzing patient's physiological data and providing monitoring, diagnosing or treating services in real time. (The hub may also provide such analysis functionality if it is a patient monitor or the like). Such wireless MBAN patient monitoring system provides a low-cost solution to extend patient monitoring services to the areas that are currently not monitored (e.g. general wards, patient homes, and the like) and allows patients to walk around in the hospital/at home without discontinuing monitoring services. This makes it possible to discharge a patient earlier from an Intensive Care Unit (ICU) or hospital, but still provide high quality care monitoring services at patient's home and this can reduce healthcare costs significantly.

To facilitate MBAN deployment, the United States Federal Communications Commission (FCC) has recently allocated a dedicated MBAN band ranging from 2360 megahertz (MHz) to 2400 MHz for MBAN services. In Europe, the European Conference of Postal and Telecommunications Administrations (CEPT) and the Electronic Communications Committee (ECC) has designated a dedicated MBAN band ranging from 2483.5 MHz to 2500 MHz for MBAN services. These bands are cleaner than the 2.4 gigahertz (GHz) industrial, scientific and medical (ISM) band, which is currently used by most wireless patient monitoring devices and many other types of wireless devices. Hence, the contemplated MBAN bands are useful for enhancing link robustness and providing medical-grade quality-of-service (QoS) in MBANs. Moreover, these bands are adjacent to the 2.4 GHz ISM band, which makes it possible to reuse low-cost, mature 2.4 GHz ISM band radios for MBAN services. Such radios include radios designed from the Institute of Electrical and Electronics Engineers (IEEE) 802.15.4 standard.

The present application provides a new and improved system and method which overcome the above-referenced problems and others.

### SUMMARY

Two major technical challenges that the MBAN design is facing are reducing sensor device power consumption (i.e. increasing their battery lives) and achieving medical-grade communication link robustness. A typical MBAN design includes a hub device and one or more sensor devices. In such a setup, each sensor just communicates with the hub and the hub is the only device that aggregates the sensor data, and there is no differentiation between sensor devices in terms of their battery/power capacities.

The hub is usually an a.c. powered device, so that battery life is not an issue. Even if the hub is battery-powered, it is usually a unit as compared with the typically miniaturized on-body MBAN sensors, and hence the battery life of the hub in such a case is long. It is recognized herein, however, that the MBAN may include other devices that are either a.c. powered or are larger devices with large batteries. For example, a mechanical ventilator machine may be included in the MBAN network, so as to supply to the hub (e.g. a patient monitor) patient data such as airway flow and pressure via the MBAN. The ventilator may also monitor or utilize data from one or more physiological sensors of the MBAN, such as an ECG. Conventionally, the ventilator would receive the ECG data indirectly via the hub.

It is recognized herein, however, that such devices are well-placed to provide data aggregation support for the hub. They are a.c. powered (or at least have large batteries), include the radio hardware for short-range MBAN communication which is prerequisite for joining the MBAN, and in some cases indirectly collect physiological data from the hub.

Thus, in embodiments disclosed herein, such a device serves as an aggregator device providing support for the hub. The short range wireless communication of the aggregator device (e.g., the ventilator) is used to directly collect sensor data that is already broadcast by the miniaturized sensors (albeit with the hub being the intended target). This data may optionally be used at the aggregator. Additionally, if the hub misses some sensor data it can request and receive that data from the aggregator device (e.g. from the ventilator). This improves robustness of the MBAN communication and reduces battery draw on the miniaturized sensor that would otherwise need to re-transmit the sensor data that was missed by the hub.

In accordance with one aspect, a Medical Body Area Network (MBAN) includes a hub device with a wireless communication transceiver. One or more sensor devices each include a physiological sensor for acquiring physiological data and a wireless communication transceiver configured to connect with the wireless communication transceiver of the hub device to wirelessly transmit acquired physiological data to the hub device. One or more aggregator devices each include a wireless communication transceiver for receiving physiological data acquired by the one or more sensor devices. The hub device is configured to respond to not wirelessly receiving a missing data portion of physiological data acquired by a sensor device by requesting and receiving the missing data portion from an aggregator device.

In accordance with another aspect, a non-transitory computer readable medium storing instructions executable by a hub device includes at least one microprocessor to perform a method for monitoring a patient using a Medical Body Area Network (MBAN). The method includes: at a hub device of the MBAN, maintaining a data types list of each type of data collected by at least one sensor device of the MBAN and an aggregator list of at least one aggregator device of the MBAN that is currently receiving one or more types of sensor data from the at least one sensor device; and, at the hub device, wirelessly receiving data from the at least one sensor device and, in response to not wirelessly receiving a missing data portion from the at least one sensor device, first requesting and receiving the missing data portion from an aggregator device listed on the aggregator list of the hub device as receiving the data type of the missing data portion and in response to not wirelessly receiving a missing data portion from the aggregator device, receiving the missing data portion from the source sensor device.

In accordance with another aspect, a Medical Body Area Network (MBAN) system for transmitting patient data is provided. The system includes at least one sensor device each including a physiological sensor for acquiring physiological data and a wireless communication transceiver. At least one aggregator device each includes a wireless communication transceiver for receiving physiological data acquired by the at least one sensor device. A hub device includes a wireless communication transceiver configured to connect with the wireless communication transceiver of the at least one sensor device to wirelessly transmit acquired physiological data to the hub device. The hub device includes at least one processor programmed to: receive a request from the at least one aggregator device to join the MBAN that the hub device is associated with; check the types of sensor data that the at least one aggregator device wants to receive; determine if the at least one aggregator device is willing to help the at least one sensor device perform retransmission when data loss happens between transmissions between the at least one sensor device and the hub device; notify the at least one aggregator device of the types of sensor data for which the aggregator device is responsible for retransmission to the hub device; receive data from the at least one aggregator device of data associated with the aggregator device and aggregating data from the at least one sensor device for which the at least one aggregator device is responsible; send a non-acknowledgment message to the at least one aggregator device when the hub device does not correctly receive sensor data from the at least one sensor device; request the sensor data from the at least one aggregator device; send a non-acknowledgment message to the at least sensor device when the hub device is notified that the aggregator device does not correctly receive the sensor data; request the sensor data from the at least one sensor device from which the missing data originates when the hub device does not correctly receive the retransmitted sensor data from the at least one aggregator device; and send an acknowledgment message to the at least one sensor device and the at least one aggregator device when the hub device correctly receives the retransmitted sensor data. One advantage resides in reducing sensor device power consumption.

Another advantage resides in increasing sensor device battery lives.

Another advantage resides in achieving improved, e.g. medical-grade, communication link robustness.

Another advantage is reduced wireless data traffic on the short-range MBAN.

Still further advantages of the present disclosure will be appreciated to those of ordinary skill in the art upon reading and understand the following detailed description. It will be appreciated that a given embodiment may attain none, one, two, more, or all of these advantages.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 illustrates a medical body area network (MBAN) according to the prior art.

FIG. 2 illustrates an MBAN solution architecture in accordance with one aspect of the present disclosure.

FIG. 3 illustrates a schematic of an MBAN of FIG. 2.

FIG. 4 illustrates a method for transmitting patient data using the MBAN of FIG. 3.

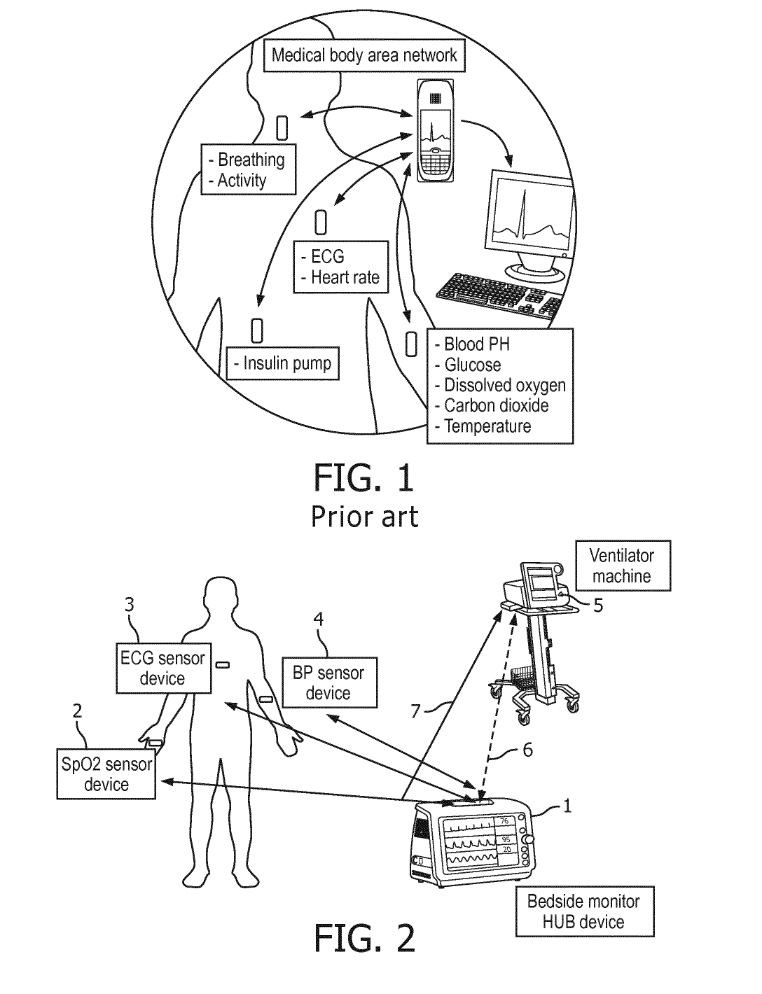

### DETAILED DESCRIPTION

To provide an integrated solution to the above-mentioned problems, more and more medical devices are connected together. Some of those medical devices are not typical “sensor” devices and they can be main, alternating-current (AC) powered or equipped with a big-capacity battery. These devices may generate their own data and/or may need to receive data from sensor devices. By leveraging these devices as additional data aggregators, there can be more than one data aggregator devices in an MBAN network and different devices can have quite different battery/power capacities.

One typical example is an MBAN patient monitoring system used in ICU, which not only includes typical on-body sensor devices, which are tiny and powered by small batteries, to capture patient physiological data, but also includes multiple main (or AC) powered bedside devices, such as a patient monitor, intravenous (IV) pump, ventilator, or anaesthesia machine. Besides the bedside monitor, ventilator machine or anaesthesia machine may also need to receive patient physiological data from on-body sensor devices. For example, the ventilator machine may need patient real-time SpO2 data to optimize its ventilator settings.

Nowadays, most of radio frequency (RF) radios can work on a wide frequency range that may cover several frequency bands. For example, Institute of Electrical and Electronics Engineers (IEEE) 802.15.4 and future IEEE 802.15.6 radios typically work in roughly the 2300-2500 megahertz (MHz) frequency range, which covers the United States MBAN band (i.e., 2360-2400 MHz), the 2.4 gigahertz (GHz) industrial, scientific and medical (ISM) band (i.e., 2400-2483.5 MHz), and the Europe MBAN band (i.e., 2483.5-2400 MHz). The 2.4 GHz ISM band is always available for MBAN operations even if it is “dirty” (i.e., more likely to have interference) compared to the MBAN bands. Hence, the 2.4 GHz ISM band opens door to operate MBANs in different bands simultaneously to offload some traffic from a channel in the MBAN bands to other channel(s) outside the MBAN bands so that the duty cycle limit can be met.

Referring to FIG. 2, an MBAN solution architecture is shown. A bedside monitor 1 and on-body sensor devices, e.g. an illustrative SpO2 sensor 2, ECG sensor 3, and blood pressure (BP) sensor 4, form a star-topology MBAN network. The bedside monitor 1 is the hub device that maintains the MBAN network and aggregates the data from all the sensor devices 2, 3, 4. Conventionally, each sensor only communicates to the hub device via MBAN to transfer its sensor data. If a data packet is lost during the transmission from a sensor to the hub, then the sensor has to retransmit the packet by itself. Due to patient body movement, the sensor-to-hub links can become weak from time to time and such retransmissions can happen frequently in reality. Those retransmissions can significantly reduce sensor device's battery life. Moreover, retransmissions performed by the same sensor may not be able to guarantee the delivery of the lost data since in many cases, the problem with the communication link between the sensor and the hub that initially caused the lost data may not change too much and still has a bad quality during retransmissions (e.g. patient body blocks the link between the sensor and the hub). To overcome this, some type of diversity scheme is needed. For example, multi-hop relay scheme is introduced in the IEEE 802.15.6, which complicates the network protocols and consume extra sensor device power.

At the same time, a ventilator machine 5 has to setup a communication link 6, either an MBAN link or other wired or wireless link, to get the SpO2 data from the patient monitor device. For example, a wired link may be provided between the ventilator and the patient monitor via an interface module. This requires extra hardware/software support and reduces system efficiency, in the sense that the SpO2 sensor data has to be transmitted two times, one from the SpO2 sensor to the patient monitor and the other from the patient monitor to the ventilator.

In general, conventional MBAN solutions are not optimized for the applications with multiple data aggregated devices (patient monitor, ventilator, and the like) since: (1) the aggregated sensor data has to be sent multiple times to get to all the aggregated data devices, which reduces system efficiency; and (2) the sensor devices with limited power capacity have to perform data retransmission when there is data loss, which reduces sensor device battery life.

With continuing reference to FIG. 2, in disclosed embodiments multiple data aggregator devices are provided. In the illustrative example, the ventilator 5 serves as a second aggregator device in addition to the hub 1. This leverages that fact that the sensors 2, 3, 4 of the MBAN are broadcasting their data to the bedside monitor or other hub device. Thus, there is no extra power consumed at the sensor (e.g. SpO2 sensor 2) if other aggregator devices, such as the illustrative ventilator machine 5, “eavesdrop” on this broadcast by also receiving the data (as diagrammatically indicated in FIG. 2 by communication path 7). This does not consume extra power at the aggregator device 5 since the aggregator does not need to receive the sensor data from the hub. In this case, if the hub 1 misses some data sent from the SpO2 sensor 2 for any reason, it can request and receive this data from the aggregator device 5. This reduces power drain on the miniaturized sensor 2, and additionally improves robustness by providing a redundant pathway for the SpO2 data to reach the hub 1. A further advantage is that when the ventilator 5 successfully receives the SpO2 data directly from the broadcast of the SpO2 sensor 2 it does not need to add this traffic to the communication link 6. In general, the disclosed approach of leveraging a.c.-powered or large battery-powered devices, and especially data consuming devices, as additional aggregators allows the MBAN to leverage the broadcasting nature of wireless communications and make other data aggregator devices also receive their desired sensor data when sensor devices transfer their data to the hub device and allow the data aggregator devices perform data retransmissions for the sensor devices to reduce sensor device power consumption.

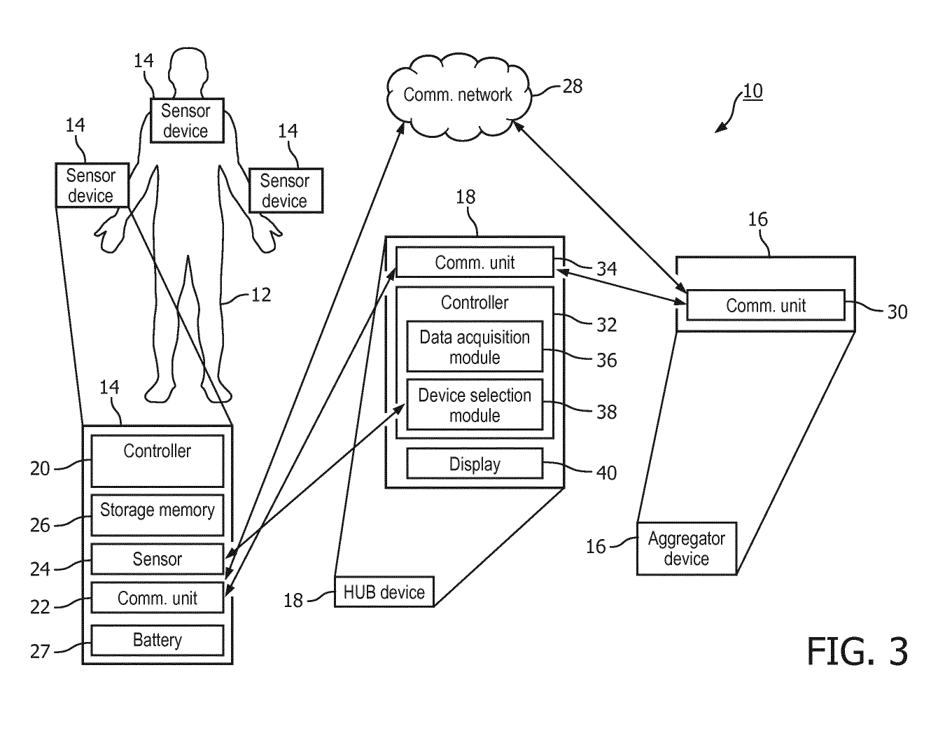

With reference to FIG. 3, in an illustrative embodiment an MBAN system or network 10 is associated with a patient 12. As shown in FIG. 3, the MBAN system 10 includes at least one sensor device 14 (more particularly three illustrative sensor devices in FIG. 3), at least one aggregator device 16, and a hub device 18. It will be appreciated that, although only one MBAN system 10 is shown in FIG. 3, an environment (such as a medical institution, a nursing home, a patient's home, and the like) can include more than one MBAN system (i.e., one MBAN system per patient 12). The sensor devices 14, the aggregator devices 16, and the hub device 18 can be in communication with each other via a wireless communication network 28 (e.g., a wireless local area network, a personal area network, Zigbee®, and the like). The sensor devices 14, the aggregator devices 16, and the hub device 18 are each described in more detail below.

The MBAN system 10 is a low-power, short-range wireless network operating in a dedicated MBAN band, such as the 2300 megahertz (MHz) to 2600 MHz band. The MBAN 10 further operates in one or more other bands, such as the 2.4 GHz ISM band, when approaching the duty cycle limit of the MBAN band. The MBAN band and the other bands are partitioned into channels managed by the hub device 18. The MBAN 10 is of any type, but typically one of an Institute of Electrical and Electronics Engineers (IEEE) 802.15.6 MBAN and an IEEE 802.15.4 MBAN.

The sensor devices 14 are configured to acquire physiological data of the patient 12, such as heart rate, respiration rate, blood pressure, electrocardiogram (ECG) signals, blood-glucose levels, oxygen-saturation levels, and so forth, in real-time and forward the data to the hub device 18 over the MBAN 10. The sensor devices 14 are typically miniature devices that are disposed on the exterior of the patient 12. For example, the sensor devices 14 can be on-body and/or wearable sensor devices. However, in some embodiments, the sensor devices 14 are additionally or alternatively disposed in the patient 12 and/or proximate to the patient.

Each of the sensor devices 14 is typically a self-contained wireless communicating device, and includes a controller 20, a wireless communication unit or transceiver 22, and at least one sensor 24 for measuring at least one physiological parameter of the patient 12. The controller 20 acquires the physiological data using the sensor 24 and transmits the acquired physiological data directly to the hub device 18 using the communication unit 22. The controller 20 typically transmits the captured physiological data upon receiving it. However, in some embodiments, the controller 20 buffers or otherwise stores the captured physiological data in at least one storage memory 26 of the sensor device 14 and transmits the buffered physiological data only when the amount exceeds a threshold. The communication unit 22 communicates with the hub device 18 over the MBAN 10. For example, the communications unit 22 is configured to operate in the frequency band of 2300 Megahertz-2600 Megahertz. The data acquired by the sensors 24 can be stored in a memory 26. In some embodiments, the sensor devices 14 are also configured to communicate with the aggregator devices 16 via the communication unit 22. In other embodiments, the sensor devices 14 are also configured to communicate with the aggregator devices 16 via a separate communication unit (not shown). The sensor device is typically battery-powered by a battery 27, and is typically miniaturized and light weight to facilitate being unobtrusive when worn by the patient. To achieve miniaturization and to lower the weight, the battery 27 is preferably a small battery and accordingly has limited energy storage. It is therefore desired to minimize power draw on the battery 27 by various mechanisms such as using low-power electronic components, and by wirelessly transmitting sensor data using as low power as possible while still achieving suitable connectivity. It will be recognized that transmitting at low power reduces the signal-to-noise ratio (SNR) and increases likelihood of missing data at the receiver (e.g. the hub 18).

The aggregator devices 16 can include a mechanical ventilator, an intravenous (IV) infusion pump, an insulin pump, an anesthesia machine, and the like. Each aggregator device typically joins the MBAN to send patient data generated at the aggregator device to the hub, and/or to receive patient data from one or more sensor devices 14 of the MBAN. The aggregator devices are preferably a.c. powered and/or powered by a relatively large battery (e.g. usually a.c. powered but with a backup battery to keep running when unplugged to move to a different location or during an a.c. power outage), so that additional power consumption at the aggregator device due to the device performing data aggregation is acceptable. Each aggregator device 16 includes a communication unit or transceiver 30 configured to communicate with the sensor devices 14, the hub device 18, or both. For example, the wireless receiving transceiver 30 is configured to receive the physiological data acquired by the one or more sensor devices 14 from the sensor devices. The receiving transceiver 30 is configured to operate in the frequency band of 2300 Megahertz-2600 Megahertz. As described in more detail below, the aggregator devices 16 are configured to receive the sensor data from the sensor devices 14, and transmit the sensor data to the hub device 18 when the hub device is unable to receive the sensor data from the sensor devices 14.

The illustrative hub device 18 is a patient monitor, such as a local bedside monitoring unit, although another type of hub device is contemplated such as a dedicated hub device mounted on an IV pole to achieve mobility. To save space and reduce the number of components, it is contemplated to integrate the hub device functionality into an IV infusion pump. The hub device 18 is disposed proximate to the patient 12, or at least within range of the low-power wireless transmissions emitted by the sensor devices 14 on the patient. As described in more detail below, the hub device 18 is configured to: (1) receive the sensor data from the sensor devices 14; and (2) request and receive the sensor data from the aggregator devices 16 when the hub device is unable to receive the data from the sensor devices 14. The hub device 18 consumes substantial power compared with the typically miniaturized sensor devices 14; accordingly, the hub device 18 is preferably a.c. powered and/or powered by a large battery (possibly serving as backup power when unplugged or during an a.c. power outage).

The illustrative hub device 18 includes a controller 32, a wireless communication unit or transceiver 34, a data acquisition module 36, and a device selection module 38. The controller 32 is programmed to control the operations of the wireless communication unit 34, the data acquisition module 36, and the device selection module 38. The wireless communication unit 34 is programmed to communicate (i.e., send messages) with the sensor devices 14 and the aggregator devices 16. It will be appreciated that the communication unit 34 is configured to operate in the frequency band of 2300 Megahertz-2600 Megahertz. The data acquisition module 36 is programmed to receive the acquired physiological data from the sensor devices 14, or from the aggregator devices 16 when the hub device 18 is unable to receive the data from the sensor devices. The device selection module 38 is programmed to determine whether to receive the physiological data from the sensor devices 14, or from the aggregator devices 16. The operation of each of these components 32-38 is described in more detail below.

The device selection module 38 of the hub device 18 is programmed to maintain a list of the aggregator devices 16 for each type of sensor data (e.g., heart rate, respiration rate, blood pressure, electrocardiogram (ECG) signals, blood-glucose levels, oxygen-saturation levels, and the like). The device list includes all the aggregator devices 16 (e.g., mechanical ventilator, an intravenous (IV) infusion pump, an insulin pump, an anesthesia machine, and the like) that are: (1) currently active in receiving such type of sensor data; and (2) willing to do retransmissions for the source sensor device 14. For each type of sensor data, the device selection module 38 also selects an aggregator device 16 from its aggregator device list as its retransmission agent. The device selection module 38 is programmed to request for retransmission from the aggregator device 16 (instead of the source sensor device 14) when a retransmission of such type of sensor data is needed.

The communication unit 34 of the hub device 18 is programmed to receive a “join” request message from at least one of the aggregator devices 16 (hereinafter also referred to as a “current” aggregator device). For example, the current aggregator device 16 is configured to notify the hub device 18 of its status and the types of sensor data (e.g. heart rate, respiration rate, blood pressure, electrocardiogram (ECG) signals, blood-glucose levels, oxygen-saturation levels, and the like) when the aggregator device 16 requests to join the MBAN network 10. In the join request message, the aggregator device 16 is configured to also indicate whether it is available to do retransmissions for the sensor data it selects to receive. The communication unit 34 of the hub device 18 is programmed to receive the join request message from the wireless receiving transceiver 30 of the aggregator device 16.

When the communication unit 34 of the hub device 18 receives the join request message from the aggregator device 16, the device selection module 38 of the hub device 18 is programmed to check the types of sensor data that the aggregator device 16 wants to receive and whether it is willing to help at least one of the sensor devices 14 perform retransmissions when a data loss happens on the link between the sensor device 14 and the hub device 18. The aggregator device 16 determines whether it is willing to serve as an aggregator for a particular sensor device or type of sensor data based on suitable criteria such as signal quality of the data signal received at the aggregator from the sensor device (this signal quality should be of some minimum level in order for the aggregator device to usefully perform the aggregation task), whether the aggregator device has sufficient processing power and data storage, and whether the aggregator device is using the sensor data itself.

If the hub device 18 decides to accept the join request from the aggregator device 16, the device selection module 38 is programmed to generate a join request response message that includes timing schedule information (e.g. when to transmit, transmission duration, order of transmission, and the like) of the sensor data that the aggregator device 16 tries to receive. The communication unit 34 of the hub device 18 is programmed to send the join request response message to the aggregator device 16. With such information, the aggregator device 16 knows the time at which the sensor data that it tries to aggregate is transmitted from the sensor devices 14 to the hub device 18 so that it can receive the aggregated sensor data directly from the sensor devices 14.

If the hub device 18 decides to accept the join request from the aggregator device 16, and the aggregator device is willing to be an retransmission agent, the device selection module 38 is programmed to update the list of aggregator devices 16 of each type of the sensor data that the aggregator devices plans to aggregate by adding the current aggregator device to the list. At the same time, the device selection module 38 is also programmed to update the retransmission agent of each updated device list, based on the link quality of the links between the aggregator devices 16 in the list and the hub device 18. Usually, the one with the best link quality will be selected as the retransmission agent. However, other selection criteria (e.g. overload, power constraints, and the like) can also be used.

If the current aggregator device 16 is selected as a retransmission agent by the hub device 18, the device selection module 38 is programmed to notify the current aggregator device 16 of the types of sensor data that such current aggregator device is responsible for retransmission (i.e., via a message transmitted from the communication unit 34).

When the current aggregator device 16 joins the MBAN 10, the aggregator device starts normal transmission/receiving of its own data. The current aggregator device 16 also receives the aggregated sensor data directly from the source sensor device 14 (when it is transmitted to the hub device 18) following the timing schedule indicated in the join request response message. As a result, when a sensor device 14 transmits its data to the hub device 18, all the aggregator devices 16 that need to aggregate such sensor data also receive the data.

In some embodiments, the data acquisition module 36 of the hub device 18 correctly receives the sensor data from the sensor devices 14. When this occurs, the data acquisition module 36 is programmed to send an acknowledgment message to acknowledge the correct reception of the data to the source sensor device 14 and the current aggregator device 16.

In other embodiments, the data acquisition module 36 of the hub device 18 correctly receives the sensor data from the sensor devices 14, while the current aggregator device 16 fails to receive the same sensor data. When this occurs, the aggregator device 16 requests the hub device 18 to retransmit the sensor data thereto. Once the communication unit 34 of the hub device 18 receives a retransmission request from the current aggregator device 16, the data acquisition module 36 is programmed to retransmit the sensor data to the current aggregator device 16 either from the MBAN link, or from other out-of-band link (e.g. wired link, other radio link).

In the further embodiments, the hub device 18 doesn't correctly receive the sensor data from the sensor device 14. When this occurs, the data acquisition module 36 is programmed to send a non-acknowledgment message to the current aggregator device 16 to indicate the failure of the transmission of the sensor device 14, and requests the aggregator device to do the retransmission. If the current aggregator device 16 receives the requested retransmission data correctly, it will start the retransmission of the missing sensor data in response to the retransmission request. If the retransmission succeeds, the data acquisition module 36 is programmed to send an acknowledgment message to the source sensor device 14 and the current aggregator device 16 to acknowledge the correct reception of the data that the missing data is retransmitted correctly.

In still further embodiments, if the current aggregator device 16 also does not receive the requested retransmission data correctly, it will reject the request from the hub device 18 with an indicator stating that it also does not have such data. Once the data acquisition module 36 receives the rejection response from the current aggregator device 16, it will send a retransmission request to the source sensor device 14 requesting for a retransmission by the source sensor data. Both the hub device 18 and the aggregator device 16 that don't correctly receive the sensor data try to receive the retransmitted data. Once the data acquisition module 36 correctly receives the retransmitted data from the source sensor device 14, it will send an acknowledgment message to the sensor device 14 to acknowledge receipt thereof. Advantageously, by allowing the aggregator devices 16 to collect the same sensor data from the sensor devices 14, the hub device 18 can consistently receive the data from either the sensor devices 14 or the aggregator devices 16, thereby preserving the integrity of the MBAN 10. In addition, the battery life of the hub device 18 is increased by saving power by requesting the sensor data from the aggregator device 16, rather than repeatedly requesting the data from the sensor devices 14 when a bad connection exists therebetween. Similarly, the power consumption by the hub device 18 is advantageously reduced.

The device selection module 38 of the hub device 18 is further programmed to update the current aggregator device 16 based on the real-time link quality information, aggregator overload information and other related information to optimize the performance of the MBAN 10.

In some embodiments, the hub device 18 can be configured as a patient monitor that includes a display 40 configured to display trend lines for physiological data acquired by the one or more sensor devices 14. In other embodiments, it will be appreciated that the communication unit 34 of the hub device 18 can include at least one of a wired communication transceiver and a WiFi communication transceiver. In this instance, the aggregator devices 16 are configured to send the missing data portion to the hub device 18 by communicating with the wired communication transceiver or the WiFi communication transceiver of the hub device. In further embodiments, it will be appreciated that the aggregator devices 16 and the hub device 18 are each alternating current-powered, thereby reducing power consumption and increasing battery life of the aggregator devices and the hub device.

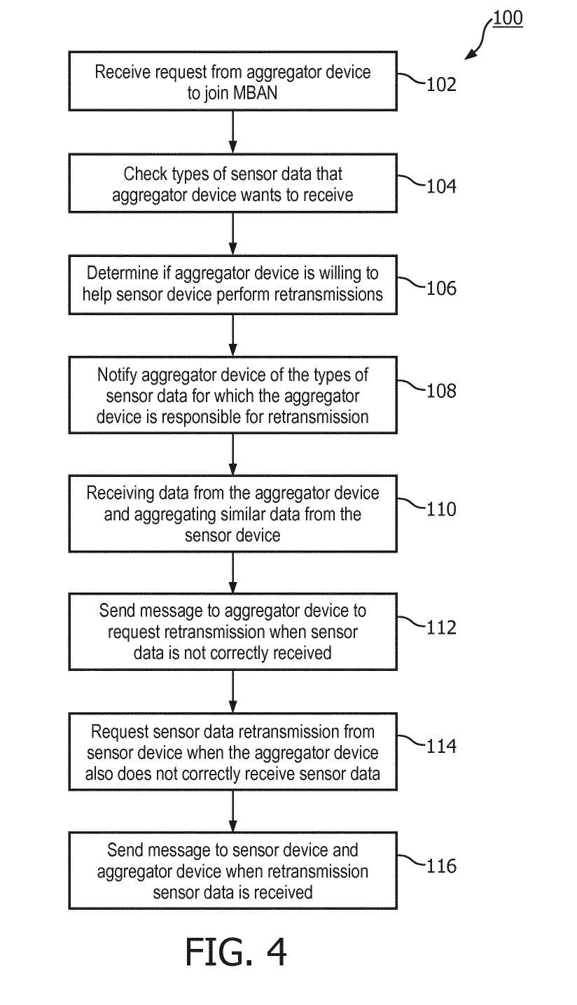

With reference to FIG. 4, a method 100 of transmitting patient data in an MBAN is provided. The method 100 includes, with a hub device 18; receive a request from the at least one aggregator device 16 to join a medical body area network (MBAN) 10 that the hub device is associated with 102; check the types of sensor data that the at least one aggregator device wants to receive 104; determine if the at least one aggregator device is willing to help at least one sensor device 14 perform retransmission when data loss happens between transmissions between the at least one sensor device and the hub device 106; notify the at least one aggregator device of the types of sensor data for which the aggregator device is responsible for retransmission to the hub device 108; receive data from the at least one aggregator of data associated with the aggregator and aggregating data from the at least one sensor device for which the at least one aggregator is responsible 110; send a non-acknowledgment message to the responsible aggregator device when the hub device does not correctly receive sensor data from the at least one sensor device to request retransmission from the aggregator device 112; request the sensor data from the source sensor device when the aggregator device also does not correctly receive the sensor data 114; and send an acknowledgment message to the at least one sensor and the at least one aggregator device when the hub device correctly receives the retransmitted sensor data 116;

As used herein, a memory includes one or more of a non-transitory computer readable medium; a magnetic disk or other magnetic storage medium; an optical disk or other optical storage medium; a random access memory (RAM), read-only memory (ROM), or other electronic memory device or chip or set of operatively interconnected chips; an Internet/Intranet server from which the stored instructions may be retrieved via the Internet/Intranet or a local area network; or so forth. Further, as used herein, a processor includes one or more of a microprocessor, a microcontroller, a graphic processing unit (GPU), an application-specific integrated circuit (ASIC), a field-programmable gate array (FPGA), and the like; a controller includes at least one memory and at least one processor, the processor executing processor executable instructions on the memory, or includes specialized hardware implementing a method; a communication unit includes a transceiver; a user input device includes one or more of a mouse, a keyboard, a touch screen display, one or more buttons, one or more switches, one or more toggles, and the like; and a display device includes one or more of a LCD display, an LED display, a plasma display, a projection display, a touch screen display, and the like.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A Medical Body Area Network, MBAN, comprising:
a hub device including a wireless communication transceiver;
and one or more sensor devices each including a physiological sensor for acquiring physiological data and a wireless communication transceiver configured to connect with the wireless communication transceiver of the hub device to wirelessly transmit acquired physiological data to the hub device; and
wherein one or more aggregator devices each including a wireless communication transceiver configured for receiving wirelessly and aggregating the acquired physiological data that are transmitted by the one or more sensor devices to the hub device;
wherein the hub device is configured to respond to not wirelessly receiving a missing data portion of the physiological data acquired by a sensor device by requesting and receiving the missing data portion from the one or more of the aggregator devices that has received the physiological data from the sensor device.

2. The MBAN of claim 1, wherein the wireless communication transceiver of the hub device and of the one or more sensor devices operates in a frequency band of 2300 Megahertz-2600 Megahertz.

3. The MBAN of claim 1, wherein the hub device further includes at least one of a wired communication transceiver and a WiFi communication transceiver, and the one or more aggregator devices are configured to send the missing data portion to the hub device by communicating with the wired communication transceiver or the WiFi communication transceiver of the hub device.

4. The MBAN of claim 1, wherein the hub device is a patient monitor comprising a display component configured to display trend lines for physiological data acquired by the one or more sensor devices.

5. The MBAN of claim 1, wherein
the at least one sensor device includes at least one of an electrocardiogram sensor, a heart rate sensor, a blood pressure sensor, a respiratory sensor, a blood-glucose sensor, and an oxygen-saturation sensor; and
the one or more aggregator devices include at least one of a mechanical ventilator, an intravenous, IV, infusion pump, an insulin pump, and an anesthesia machine.

6. The MBAN of claim 1, wherein the hub device is alternating current-powered and the one or more aggregator devices are each alternating current-powered.

7. A non-transitory computer readable medium storing instructions which when executed by the hub device of the MBAN of claim 1 cause it to receive wirelessly the acquired physiological data from the one or more sensor devices, and causing it to respond to not wirelessly receiving a missing data portion of the physiological data acquired by a sensor device by requesting and receiving the missing data portion from the aggregator device or from one of the aggregator devices that has received the physiological data from the sensor device.

8. The non-transitory computer readable medium of claim 7, storing instructions executable by the hub device to:
receive a request to join the MBAN as an aggregator from the at least one aggregator device that the hub device is associated with;
check the types of sensor data that the at least one aggregator device wants to receive;
notify the at least one aggregator device of the types of sensor data for which the aggregator device is to be responsible for retransmission to the hub device; and
receive data from the at least one aggregator device and aggregate data from the at least one sensor device for which the at least one aggregator device is responsible.

9-20. (canceled)

21. The MBAN of claim 1, wherein the hub device includes at least one processor programmed to:
receive a request from the at least one aggregator device to join the MBAN that the hub device is associated with;
check the types of sensor data that the at least one aggregator device wants to receive;
determine if the at least one aggregator device is willing to help the at least one sensor device perform retransmission when data loss happens in transmissions between the at least one sensor device and the hub device;
notify the at least one aggregator device of the types of sensor data for which the aggregator device is to be responsible for retransmission to the hub device;
receive data from the at least one sensor device for which the at least one aggregator device is responsible;
request sensor data to be retransmitted from the at least one aggregator device, when the sensor data is not correctly received;
request the sensor data to be retransmitted from the at least one sensor device when the hub device is notified that the aggregator device also does not correctly receive the sensor data;
and send an acknowledgment message to the at least one sensor device and the at least one aggregator device when the hub device correctly receives the retransmitted sensor data.

22. The MBAN system of claim 21, wherein the at least one processor is further programmed to:
transmit, to the at least one aggregator device, a timing schedule of the sensor data that the at least one aggregator device is to try to receive, when the hub device accepts the request from the at least one aggregator device to join the MBAN system.

23. The MBAN system of claim 22, wherein the at least one processor is further programmed to:
maintain a list of each type of data collected by at least one sensor device and at least one aggregator device that is currently receiving each type of sensor data from the at least one sensor device; and
update the list of each type of the sensor data that the at least one aggregator device plans to aggregate by adding the at least one aggregator device to the list when the hub device accepts the request from the at least one aggregator device to join the MBAN system when the at least one aggregator device is willing to be a retransmission agent.

24. The MBAN system of claim 21, wherein the at least one processor is further programmed to:
send an acknowledgment message to the at least one sensor device or the at least one aggregator device when the hub device correctly receives sensor data.

25. The MBAN system of claim 24, wherein the at least one processor is further programmed to:
receive a retransmission request from the at least one aggregator device when the hub device correctly receives the sensor data from the at least one sensor device and the at least one aggregator device fails to receive the same sensor data; and
retransmit the received sensor data to the at least one aggregator device.

26. A method of using an MBAN comprising:
a hub device including a wireless communication transceiver;
and one or more sensor devices each including a physiological sensor for acquiring physiological data and a wireless communication transceiver configured to connect with the wireless communication transceiver of the hub device to wirelessly transmit acquired physiological data to the hub device; and
wherein the MBAN comprises one or more aggregator devices each including a wireless communication transceiver for receiving wirelessly and aggregating the acquired physiological data that are transmitted by the one or more sensor devices to the hub device;
the method comprising the hub device responding to not wirelessly receiving a missing data portion of the physiological data acquired by a sensor device by requesting and receiving the missing data portion from the aggregator device or from one of the aggregator devices that has received the physiological data from the sensor device.

27. The non-transitory computer readable medium of claim 7, storing instructions executable by the hub device to:
maintain a data types list of each type of data collected by at least one sensor device of the MBAN and an aggregator list of at least one aggregator device of the MBAN that is currently receiving one or more types of sensor data from the at least one sensor device; and
request and receive the missing data portion from an aggregator device listed on the aggregator list of the hub device as receiving the data type of the missing data portion.
